(19) United States
(12) Patent Application Publication
(10) Pub. No.: US 2021/0346589 A1
(43) Pub. Date: Nov. 11, 2021

(54) MICRO BALLOON-ON-TUBE WOUND FILLER

(71) Applicant: KCI Licensing, Inc., San Antonio, TX (US)

(72) Inventors: Timothy Mark ROBINSON, Shillingstone (GB); Christopher Brian LOCKE, Bournemouth (GB)

(21) Appl. No.: 17/285,320

(22) PCT Filed: Oct. 11, 2019
(86) PCT No.: PCT/US2019/055872

§ 371 Date: Apr. 14, 2021

Related U.S. Application Data
(60) Provisional application No. 62/745,782, filed on Oct. 15, 2018.

(51) Int. Cl. A61M 1/00; B29D 23/00

(52) U.S. Cl. CPC A61M 1/912; A61M 1/84; B29D 23/00; B29K 2101/12; A61M 2207/00; A61M 1/85; A61M 2205/0216

(57) ABSTRACT

A dressing, system, and method for use with negative-pressure treatment is described. The dressing includes a tube formed from a non-porous material. The tube can have a first end, a second end, a lumen extending from the first end to the second end, and a wall surrounding the lumen. At least one blister is formed in the wall of the tube, the at least one blister proximate to the second end of the tube. At least one aperture is formed in the wall of the second end of the tube. The at least one aperture is configured to provide fluid communication across the wall from the lumen to an exterior of the tube.

### TECHNICAL FIELD

The invention set forth in the appended claims relates generally to tissue treatment systems and more particularly, but without limitation, to systems, dressings, and wound fillers for negative-pressure tissue treatment and instillation treatment, and methods of using systems, dressings, and wound fillers for negative-pressure tissue treatment and instillation treatment.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and instillation therapy are widely known, improvements to therapy systems, components, and processes may benefit healthcare providers and patients.

### BRIEF SUMMARY

New and useful systems, apparatuses, and methods for wound fillers in a negative-pressure therapy environment are set forth in the appended claims. Illustrative embodiments are also provided to enable a person skilled in the art to make and use the claimed subject matter.

For example, a dressing for use with negative-pressure treatment is described. The dressing can include a tube formed from a non-porous material. The tube can have a first end, a second end, a lumen extending from the first end to the second end, and a wall surrounding the lumen. At least one blister can be formed in the wall of the tube, the at least one blister proximate to the second end of the tube. At least one aperture can be formed in the wall of the second end of the tube. The at least one aperture can be configured to provide fluid communication across the wall from the lumen to an exterior of the tube.

More generally, a method of manufacturing a dressing is described. A conduit can be formed from a non-porous material. The conduit can have a proximal end, a distal end, a lumen extending from the proximal end to the distal end, and a wall surrounding the lumen. At least one bubble can be formed in the wall of the conduit, the at least one bubble proximate to the distal end of the conduit. At least one opening can be formed in the wall of the distal end of the conduit. The at least one opening can be configured to provide fluid communication across the wall from the lumen to an exterior of the conduit.

In some embodiments, the conduit can be extruded and cooled. The conduit can be placed in a mold, heated, and a gas can be applied to the lumen of the conduit to expand the conduit to fill the mold. In some embodiments, a plurality of conduits can be formed, and a plurality of mats can be formed. At least two of the plurality of conduits can be coupled to each other with a mat of the plurality of mats. A plurality of perforations can be formed in the plurality of mats. The plurality of perforations can be aligned to form tear lines between adjacent conduits of the plurality of conduits. A film can be coupled to the bubbles. For example, the film, having a plurality of perforations, can be laminated to the bubbles. In some embodiments, the plurality of conduits and the film can be co-extruded.

Alternatively, other example embodiments may describe a dressing for use with negative-pressure treatment. The dressing can have a tube formed from a non-porous material. The tube having a first end, a second end, a lumen extending from the first end to the second end, and a wall surrounding the lumen. The tube can have at least one blister in the wall of the tube, the at least one blister proximate to the second end of the tube. The tube can have at least one aperture in the wall of the second end of the tube. The at least one aperture is configured to provide fluid communication across the wall from the lumen to an exterior of the tube. The dressing can be formed by: extruding the tube and cooling the tube. The extruded and cooled tube can be placed in a mold, and the tube can be heated. A gas can be applied to the lumen of the tube, and the tube can be inflated to fill the mold and form the blister.

In some embodiments, a plurality of tubes and mats can be extruded. At least two of the plurality of tubes can be coupled to each other with a mat of the plurality of mats; and a plurality of perforations can be formed in the plurality of mats. The plurality of perforations can be aligned to form tear lines between adjacent tubes of the plurality of tubes. In some embodiments, a film can be coupled to the blisters, for example, by laminating a film to the blisters. The film can have a plurality of fenestrations. In some embodiments, the plurality of tubes and the film can be co-extruded.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a perspective view illustrating additional details of example wound fillers that may be associated with some embodiments of the therapy system of FIG. 1;

FIG. 3 is a sectional view taken along line 3-3 of FIG. 2 illustrating additional details that may be associated the wound filler of FIG. 2;

FIG. 4 is a perspective view illustrating additional details of another example wound filler that may be associated with some embodiments the therapy system of FIG. 1;

FIG. 5 is a perspective view illustrating additional details of another example wound filler that may be associated with some embodiments the therapy system of FIG. 1;

FIG. 6 is a perspective view illustrating additional details of another example wound filler that may be associated with some embodiments the therapy system of FIG. 1;

FIG. 7 is a perspective view illustrating additional details of another example wound filler that may be associated with some embodiments the therapy system of FIG. 1;

FIG. 8 is a perspective view illustrating additional details of another example wound filler that may be associated with some embodiments the therapy system of FIG. 1;

FIG. 9 is a sectional view taken along line 9-9 of FIG. 8 illustrating additional details that may be associated with the wound filler of FIG. 8;

FIG. 10 is a perspective view illustrating additional details of another example wound filler that may be associated with some embodiments of the therapy system of FIG. 1; and

FIG. 11 is a plan view illustrating additional details of the wound filler of FIG. 10 that may be associated with some example embodiments.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

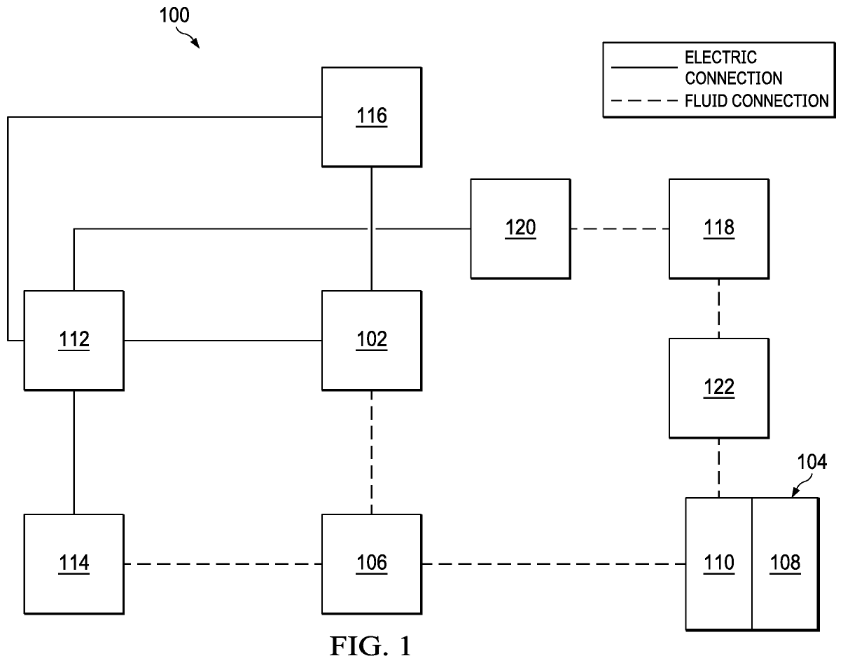

FIG. 1 is a simplified functional block diagram of an example embodiment of a therapy system 100 that can provide negative-pressure therapy with instillation of topical treatment solutions to a tissue site in accordance with this specification. The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumens or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Tex.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 114 and a second sensor 116 coupled to the controller 112.

The therapy system 100 may also include a source of instillation solution. For example, a solution source 118 may be fluidly coupled to the dressing 104, as illustrated in the example embodiment of FIG. 1. The solution source 118 may be fluidly coupled to a positive-pressure source such as a positive-pressure source 120, a negative-pressure source such as the negative-pressure source 102, or both in some embodiments. A regulator, such as an instillation regulator 122, may also be fluidly coupled to the solution source 118 and the dressing 104 to ensure proper dosage of instillation solution (e.g. saline) to a tissue site. For example, the instillation regulator 122 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 118, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 114 and the second sensor 116, are generally known in the art as any apparatus operable to detect or measure a physical phenomenon or property, and generally provide a signal indicative of the phenomenon or property that is detected or measured. For example, the first sensor 114 and the second sensor 116 may be configured to measure one or more operating parameters of the therapy system 100. In some embodiments, the first sensor 114 may be a transducer configured to measure pressure in a pneumatic pathway and convert the measurement to a signal indicative of the pressure measured. In some embodiments, for example, the first sensor 114 may be a piezo-resistive strain gauge. The second sensor 116 may optionally measure operating parameters of the negative-pressure source 102, such as a voltage or current, in some embodiments. Preferably, the signals from the first sensor 114 and the second sensor 116 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites and subcutaneous tissue sites. In these examples, the tissue interface 108 may fill the wound and be referred to as a wound filler. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Exopack Advanced Coatings, Wrexham, United Kingdom, now Coveris™ Advanced Coatings. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 114. The controller 112 may also control the operation of one or more components of the therapy system 100 to manage the pressure delivered to the tissue interface 108. In some embodiments, the controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

Some tissue interfaces are porous and able to manifold both positive pressure and negative pressure. Manifolding both positive pressure and negative pressure permits the tissue interface to transport fluids to and from the tissue site. However, a porous tissue interface, such as gauze, may suffer from ingrowth of tissue. Ingrowth of tissue or tissue ingrowth can refer to tissue growing into the pores of the tissue interface as the tissue site heals. Tissue ingrowth can cause pain or discomfort to a patient, particularly when the tissue interface is removed from the tissue site. In some cases, a tissue interface can become so filled with healed tissue that portions of the tissue interface can remain in the tissue site if the tissue interface is removed. This can occur where the tissue site is fast granulating or the tissue interface is left in place at the tissue site for greater than the prescribed time period, for example, three days. A non-adherent layer, such as a silicone gel coated mesh or a perforated film, can be disposed between the tissue interface and the tissue site. The non-adherent layer can block granulating tissue from growing into the tissue interface. However, adding a non-adherent layer to the dressing can increase the complexity of treating the tissue site.

Other tissue interfaces can cause damage to periwound areas. For example, a tissue interface having rough surfaces adjacent to a periwound area may contact and damage periwound tissue. Some tissue interfaces may draw fluid into contact with the periwound tissue. In some instances, the fluid may be held in contact with the periwound tissue, increasing the likelihood that the periwound tissue may macerate.

To appropriately treat a tissue site, the tissue interface may require sizing when the tissue interface is placed at or in the tissue site. Sizing can refer to the manipulation of the tissue interface through compression, cutting, or addition of further tissue interfaces. Sizing of a tissue interface enables the tissue interface to appropriately fit the tissue site for the particular therapy being provided. Some tissue interfaces may shed particles or fibers when the tissue interface is sized to fit a particular tissue site. The particles or fibers can be inadvertently left in the tissue site, potentially causing pain, discomfort, and in rare instances healing complications for the patient.

Many tissue interfaces are also opaque. An opaque tissue interface can inhibit examination of the tissue site during therapy. Thus, to properly examine the tissue site during the therapy process, the dressing, including the cover and the tissue interface, are removed and replaced. Frequent replacement of the dressing may cause irritation of the periwound tissue and increase the costs of therapy. For all of the foregoing reasons, there has been a long-felt need for a tissue interface that does not require an interposing non-adherent interface to reduce tissue ingrowth, reduces damage to periwound areas, is still able to generate apposition forces, and create micro and macrostrain at a tissue site.

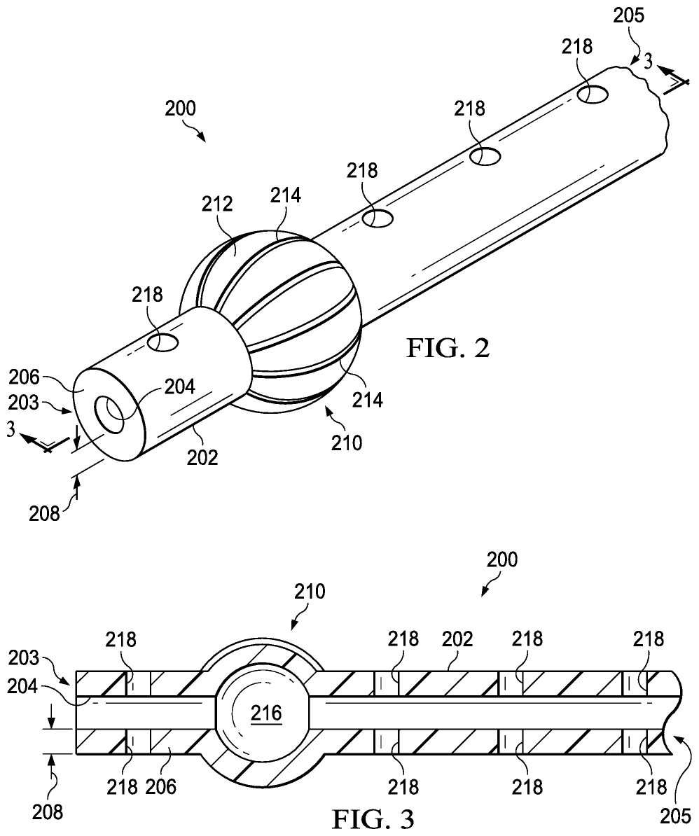

FIG. 2 is a perspective view illustrating additional details of an embodiment of the tissue interface 108, for example, a wound filler 200. The wound filler 200 may provide a smooth porous surface that can promote granulation. The wound filler 200 can act as a manifold for both positive pressure and negative pressure while reducing instances of ingrowth. Furthermore, the wound filler 200 can also be covered with a fenestrated or perforated film, providing additional benefits for healing of the tissue site.

The wound filler 200 can be a fluid conductor, such as a conduit or a tube 202. The tube 202 may have a first end or distal end 203 and a second end or proximal end 205. The tube 202 may have lumen, such as a bore 204. In some embodiments, the tube 202 may be a cylindrically shaped object having a substantially circular cross section. The tube 202 may have the bore 204 formed coaxially with the tube 202. The bore 204 may form a wall 206 having a wall thickness 208. The wall 206 may be annular, and the wall thickness 208 may be substantially uniform. In some embodiments, the bore 204 may have an average effective diameter between about 0.1 millimeter (“mm”) and about 2.0 mm, forming an inner diameter of the tube 202. An average effective diameter is the diameter of a circle or a sphere having the same area or volume as the non-circular or non-spherical object. In some embodiments, the wall thickness 208 may be between about 0.05 mm and about 2 mm. The tube 202 may have an average effective diameter between about 0.3 mm and about 6 mm. In other embodiments, the tube 202 may be a rectangular body having a square or rectangular cross section, a pyramid-shaped body having a triangular cross section, or have an amorphous shape having an amorphous cross section. The tube 202 can communicate fluid between the proximal end 205 of the tube 202 and the distal end 203 of the tube 202 through the bore 204. In some embodiments, the tube 202 may have a length of about 30 centimeters (“cm”). In other embodiments, the tube 202 may have a length greater than about 30 cm. For example, the tube 202 may be provided in lengths permitting the tube 202 to be rolled onto a cylindrical member for storage.

In some embodiments, a plurality of perforations 218 can be formed in the tube 202. The plurality of perforations 218 can be axially disposed along the length of the tube 202. In some embodiments, the plurality of perforations 218 are equidistantly spaced along the length of the tube 202. For example, adjacent perforations 218 may have a pitch, a distance between respective centers, of about 5 mm. The perforations 218 can extend an entire length of the tube 202. In other embodiments, the plurality of perforations 218 may have a non-regular spacing. In some embodiments, each perforation 218 may have a corresponding perforation 218 disposed radially across from the subject perforation 218. In other embodiments, each perforation 218 may have a non-straight angle between axially adjacent perforations 218. Each perforation may have a diameter between about 0.2 mm and about 3 mm. In some embodiments, each perforation 218 may be circular. In other embodiments, each perforation 218 may be triangular, square, ovular, or amorphous shaped having an average effective diameter between about 0.2 mm and about 3 mm.

A balloon, micro-balloon, bubble, or blister 210 may be formed on the tube 202. In some embodiments, the blister 210 can be positioned proximate to the distal end 203 of the tube 202. The blister 210 may be spaced from the distal end 203. In some embodiments, an outer periphery of the blister 210 proximate to the distal end 2013 may be about 1 cm from the distal end 203. In other embodiments, the blister 210 may be positioned at the distal end 203. The tube 202 may have more than one blister 210. For example, the tube 202 may have a blister 210 positioned proximate to the proximal end 205; between the distal end 203 and the proximal end 205; or at multiple locations from the proximal end 205 to the distal end 203. The blister 210 may have an overall average effective diameter between about 1 mm and about 5 mm. The blister 210 may be spherical. In other embodiments, the blister 210 may be hemispherical, polygonal, or cone-like. In some embodiments, the blister 210 may have a surface 212. In some embodiments, the surface 212 may be smooth. In other embodiments, the surface 212 may have a plurality of grooves 214. The grooves 214 may be parallel to each other or non-parallel. In some embodiments, each groove 214 may have a width between about 50 microns and about 300 microns. The grooves 214 may have a pitch equal to about the width of each groove 214. In other embodiments, the grooves 214 may have a pitch up to about twice the width of each groove 214. The grooves 214 may provide manifolding, fluid management, and microstrain if in contact with a surface of a tissues site.

In some embodiments, the tube 202 may be formed from a non-porous material. The tube 202 may be formed from thermoplastic. For example, the tube 202 may be formed from polythene, polyester, or polyurethane. In other embodiments, the tube 202 may be formed from a thermoplastic elastomer, such as, styrene ethylene butadiene styrene (SEBS), or an elastomeric, such as silicone. In some embodiments, the tube 202 may be transparent, opaque, or have a transparency between fully transparent and fully opaque. Preferably, the tube 202 may be transparent or translucent. The tube 202 may have a hue. For example, the tube 202 may be red, green, blue, or a combination of red, green, and blue.

FIG. 3 is a sectional view of the wound filler 200 taken along line 3-3, illustrating additional details that may be associated with some embodiments. The blister 210 may have an interior space 216. The interior space 216 may be in fluid communication with the bore 204 of the tube 202. The interior space 216 can provide a light-weight filler and aid in flexibility. Preferably, the interior space 216 may be at least 20% of the volume of the wound filler 200. Each perforation 218 of the plurality of perforations 218 may extend through the wall 206. The plurality of perforations 218 may be evenly spaced along a length of the tube 202 and may be circumferentially spaced around the tube 202. In other embodiments, the plurality of perforations 218 may be disposed along the length and circumference of the tube 202 without a pattern. Each of the plurality of perforations 218 may provide fluid communication across the wall 206 between the bore 204 and the environment surrounding the wound filler 200.

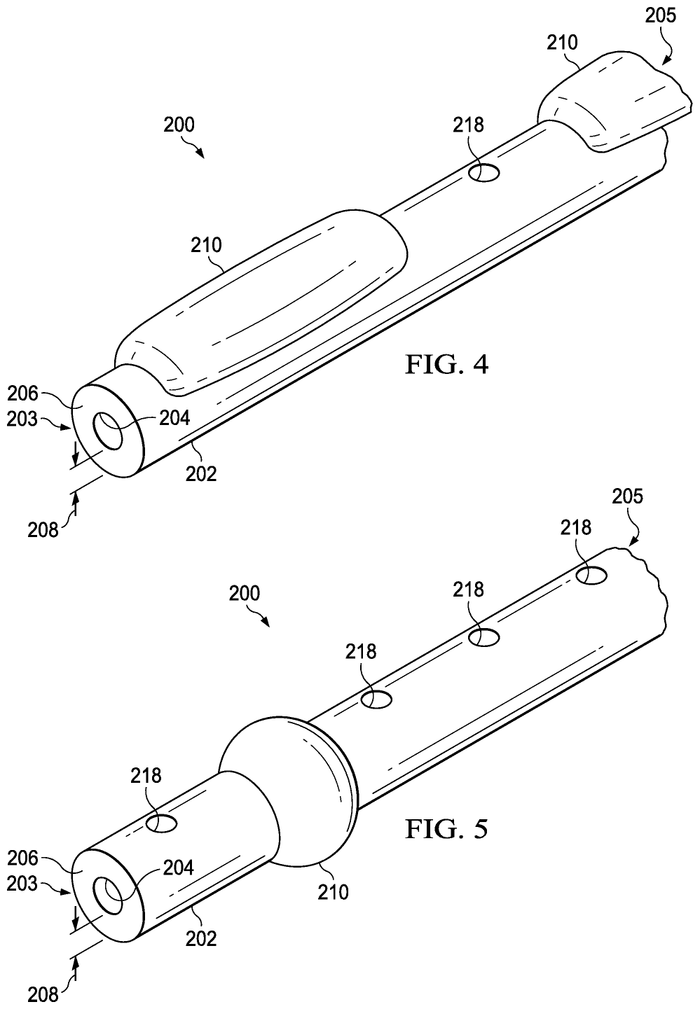

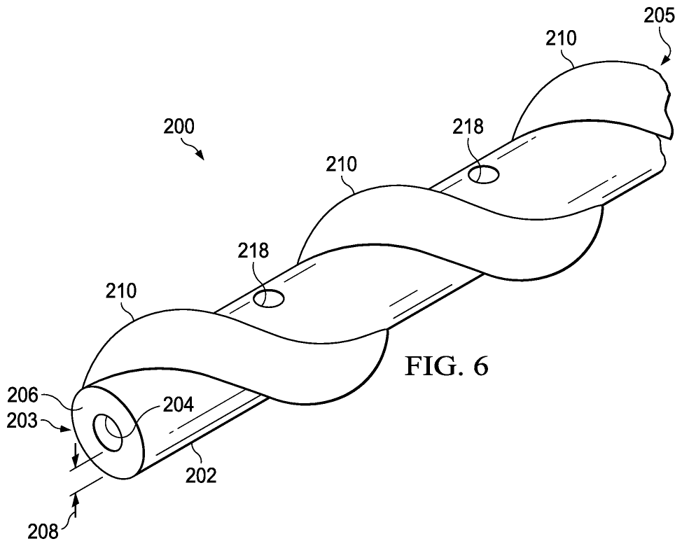

The tube 202 may be manufactured by extruding the tube 202. For example, a thermoplastic can be pushed through a die having the desired shape of the tube 202. Often, the extrusion process can heat the material, and the extruded piece can be cooled to solidify the piece. After extrusion and cooling, the tube 202 can be placed into a mold. The mold may be formed having the shape of the blister 210. A portion of the tube 202 can be heated and a gas can be injected into the bore 204. The gas can expand the portion of the tube 202 within the mold, creating the blister 210. In other embodiments, the tube 202 can be subject to a vacuum after placement in the mold. The vacuum may draw the heated portion of the tube 202 into the mold to form the blister 210. In other embodiments, the tube 202 may be extruded and cooled. The tube 202 may be pressurized and a portion of the tube 202 may be heated with a laser. For example, a pressurized gas can be applied to the bore 204 of the tube 202. A laser can then be focused on an area of the tube 202 where it is desired to form a blister 210. The laser may create a softened zone that can expand into the blister 210 due to the pressurization of the bore 204. In some embodiments, the laser can be used to create blisters 210 having longitudinal, circumferential, and spiral patterns. For example, FIG. 4 is a perspective view illustrating additional details of another embodiment of the tube 202. The tube 202 or the laser may be moved parallel to an axis of the tube 202 as the tube 202 is heated with the laser to form a blister 210 having a longitudinal shape parallel to the axis of the tube 202. FIG. 5 is a perspective view illustrating additional details of another embodiment of the tube 202. The tube 202 can be rotated on its axis while being heated with the laser to form a blister 210 that circumscribes the tube 202. FIG. 6 is a perspective view illustrating additional details of another embodiment of the tube 202. The tube 202 may be rotated on its axis while the tube 202 or the laser is moved axially and the tube 202 is heated. The relative motion can produce a blister 210 that winds spirally around the tube 202.

In some embodiments, a blowing agent can be added to the material of the tube 202 prior to extruding the tube 202. Preferably, a blowing agent having a biocompatible residue or that leaves no residue may be used. For example, the blowing agent can be solid carbon dioxide; a nitrogen liberating substance such as amines, azides, and carbamates; a low boiling point liquid; a dissolved gas, such as nitrogen or carbon dioxide; or a polymer microsphere, such as Expancel®. After extrusion, the tube 202 can be rapidly cooled. The tube 202 can be placed in a mold capable of heating the tube 202 in small discrete zones. Heating the small discrete zones can trigger the blowing agent to expand, forming the blister 210. In some embodiments, the tube 202 may be crimped, creased, kinked, or pinched along its length. The crimps can be positioned to increase flexibility of the tube 202. In some embodiments, the crimps can be positioned about every 5 cm.

In operation, the wound filler 200 can be disposed in a tissue site. For example, the tube 202 having the blister 210 can be inserted into a tissue site to substantially fill the tissue site. In some embodiments, multiple would fillers 200 can be disposed in the tissue site. For example, multiple wound fillers 200 can be disposed in the tissue site, substantially filling the tissue site. The surface 212 can be positioned in contact with the tissue site. If the surface 212 is formed with the grooves 214, the grooves 214 may be in contact with the tissue site. An end of the tube 202 opposite the blister 210 can be fluidly coupled to the negative-pressure source 102 or the solution source 118, or both. In other embodiments, the wound filler 200 may not be directly coupled to the negative-pressure source 102 or the solution source 118. The cover 110 can be positioned over the tube 202 and sealed to periwound surrounding the tissue site to form a sealed therapeutic environment. The negative-pressure source 102 or the solution source 118 can be operated to draw fluid from or supply fluid to the tissue site via the tube 202. Fluid may flow from the tissue site through the perforations 218 into the bore 204 and to the vacuum source. Alternatively, fluid may flow from the bore 204 through the perforations 218 and into the tissue site. The blister 210 may maintain separation between an outer surface of the tube 202 and the tissue site, providing free space for the flow of fluid between the tube 202 and the tissue site. In some embodiments, the free space provided by the blister 210 may improve the manifolding behavior of the wound filler 200. For example, the application of negative pressure to the tube 202 may draw tissue adjacent to the tube 202. The blisters 210 can hold the remaining surface of the tube 202 apart from the tissue to provide a flow path between the tissue and the surface of the tube 202.

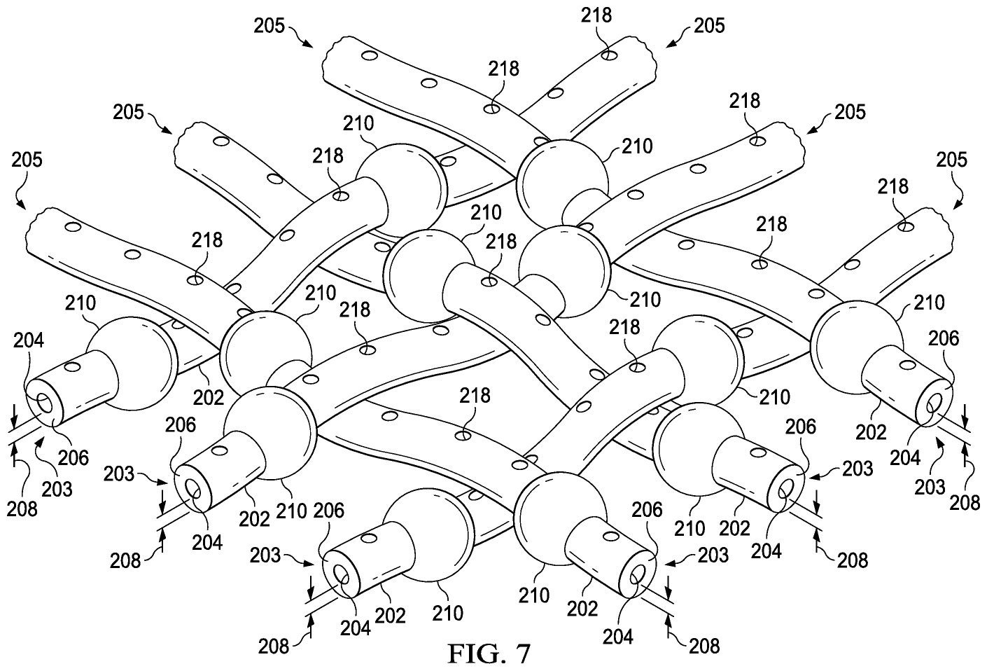

FIG. 7 is a perspective view illustrating additional details that may be associated with some embodiments. In some embodiments, a plurality of tubes 202 may be woven into a mat. The mat can be disposed within the tissue site. The mat of tubes 202 can be covered by the cover 110 and fluidly coupled to the negative-pressure source 102. Fluid may be drawn from the tissue site through the perforations 218 of the mat of tubes 202 into the bores 204 for removal and storage. In some embodiments, a plurality of mats may be used.

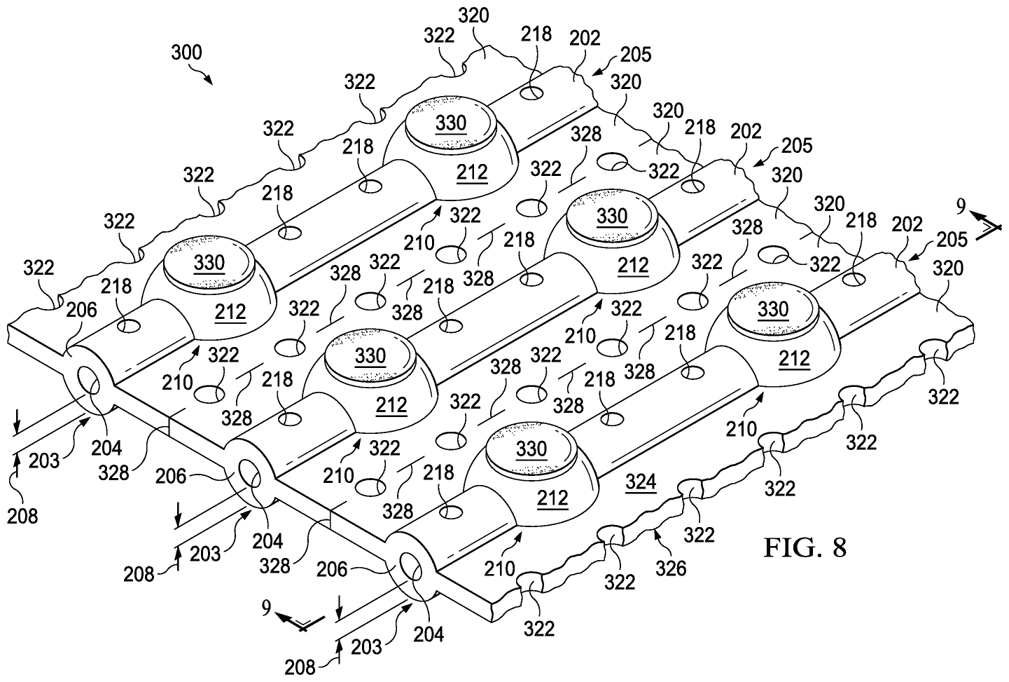

FIG. 8 is a perspective view illustrating additional details of another embodiment of the tissue interface 108. The tissue interface 108 can be a wound filler 300. The wound filler 300 can include a plurality of the tubes 202. Each of the tubes 202 may have a plurality of blisters 210. In some embodiments the blisters 210 may all have a same diameter. In other embodiments, the blisters 210 may have different diameters. For example, a first blister 210 may have a first average effective diameter, a second blister 210 may have a second average effective diameter, and a third blister 210 may have a third average effective diameter. The first average effective diameter, the second average effective diameter, and the third average effective diameter may be different from each other. Each blister 210 may be aligned with adjacent blisters 210 on adjacent tubes 202. In other embodiments, each blister 210 may not be aligned with adjacent blisters 210 on adjacent tubes 202. In some embodiments, the blisters 210 may be staggered from one another. For example, adjacent blisters 210 may have a pitch of about a diameter of the blister 210.

In some embodiments, each tube 202 of the plurality of tubes 202 may be co-planar, forming a sheet of the tubes 202. In other embodiments, each tube 202 of the plurality of tubes 202 may not be co-planar. Each of the tubes 202 may be joined to an adjacent tube 202 by a webbing, a mat, or a membrane 320. The membrane 320 can have a length about equal to a length of the tube 202, and a width between adjacent tubes 202 equal to about the outer diameter of the tube 202. In some embodiments, the membrane 320 may have a thickness of at least 10 microns. Each membrane 320 may have a plurality of fenestrations or apertures 322. The plurality of apertures 322 may provide fluid communication across the membrane 320 from a first side 324 of the membrane to a second side 326 of the membrane 320. In some embodiments, the apertures 322 may be aligned so that centers of each aperture 322 are equidistantly spaced between adjacent tubes 202. In other embodiments, the apertures 322 may not be aligned. In some embodiments, each aperture 322 may have an average effective diameter between about 0.2 mm and about 3 mm and a pitch between adjacent apertures 322 of about 3 mm. Each membrane 320 joining adjacent tubes 202 may also have a plurality of perforations forming a tear line 328. The tear line 328 may be equidistant from adjacent tubes 202. The tear line 328 may permit adjacent tubes 202 to be separated from each other, for example, for sizing purposes.

In some embodiments, the surface 212 of each blister 210 may be smooth. For example, the surface 212 may have an SPI Finish of C1. An adhesive 330 may be disposed on the surface 212 of each blister 210. In some embodiments, the adhesive 330 may cover the entirety of the surface 212. In other embodiments, the adhesive 330 may cover less than about 30% of the surface 212. The adhesive 330 may be disposed the surface 212 so that the adhesive 330 of each blister 210 is co-planar. The plurality of adhesives 330 may occupy a plane that is parallel to a plane containing the membrane 320.

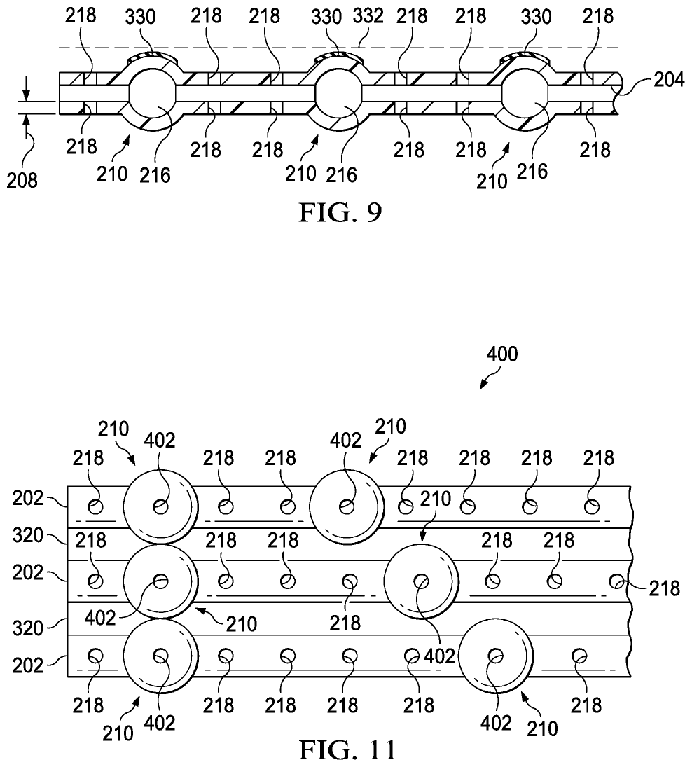

FIG. 9 is a sectional view of the wound filler 300 taken along line 9-9 of FIG. 8, illustrating additional details that may be associated with some embodiments. A film 332 can be coupled to the adhesive 330. The film 332 may have a thickness of about 25 microns. In some embodiments, the film 332 may be kept taut between adjacent blisters 210 and tubes 202, forming a cavity between the membrane 320, the film 332, and the tubes 202. In other embodiments, the film 332 may contact the membrane 320 between adjacent tubes 202. The film 332 can be fenestrated or perforated to permit fluid and pressure communication across the film 332. The fenestrations can be about 3 mm long and have a pitch between adjacent fenestrations of about 3 mm. In some embodiments, the fenestrations may be aligned with the tear lines 328 of the membrane 320 to assist with sizing of the wound filler 300. In some embodiments, the film 332 may be formed from thermoplastic. For example, the film 332 may be formed from polythene, polyester, or polyurethane. In other embodiments, the film 332 may be formed from a thermoplastic elastomer, such as, styrene ethylene butadiene styrene (SEBS), or an elastomeric, such as silicone. In some embodiments, the film 332 may be transparent, opaque, or have a transparency between fully transparent and fully opaque. The film 332 may have a hue. For example, the film 332 may be red, green, blue, or a combination of red, green, and blue.

The wound filler 300 may be manufactured by co-extruding and cooling the tubes 202 and the membranes 320 as described above with respect to the wound filler 200. The wound filler 300 can be placed into a mold. The mold may be formed having the shape and spacing of the blisters 210. A portion of the wound filler 300 can be heated and a gas can be injected into the bores 204. The gas can expand the portion of each tube 202 within the mold, creating the blisters 210. The membranes 320 can then be perforated to form the apertures 322 and the tear lines 328. The adhesive 330 and the film 332 can be laminated or extruded onto the blisters 210.

In operation, the wound filler 300 may be provided in a roll. A user may cut a length of the wound filler 300 as required to fill the tissue site. In other embodiments, the wound filler 300 may be provided in pre-cut lengths, for example, between about 10 cm to about 30 cm. If necessary, one or more tubes 202 can be removed from the wound filler 300 by tearing a tube 202 from an adjacent tube 202 along a tear line 328. The wound filler 300 can be pushed directly into a tissue site. In other embodiments, the wound filler 300 can be wound in a loose coil and placed into the tissue site. For a shallow tissue site, a single layer of the wound filler 300 may be used. The wound filler 300 and the tissue site can be covered with a cover 110 to form a sealed therapeutic environment. The sealed therapeutic environment can be fluidly coupled to the negative-pressure source 102. The surface 212 of the blisters 210 may provide free space between the wound filler 300 and the tissue site. The free space may provide a pathway for fluid communication between the tissue site and the wound filler 300. Fluid may flow from the tissue site through the free space into the perforations 218 and then into the bore 204. There, the fluid may be communicated to the container 106 for storage. In some embodiments having multiple wound fillers 300, fluid may flow between adjacent wound filers 300 across the fenestrated film 332.

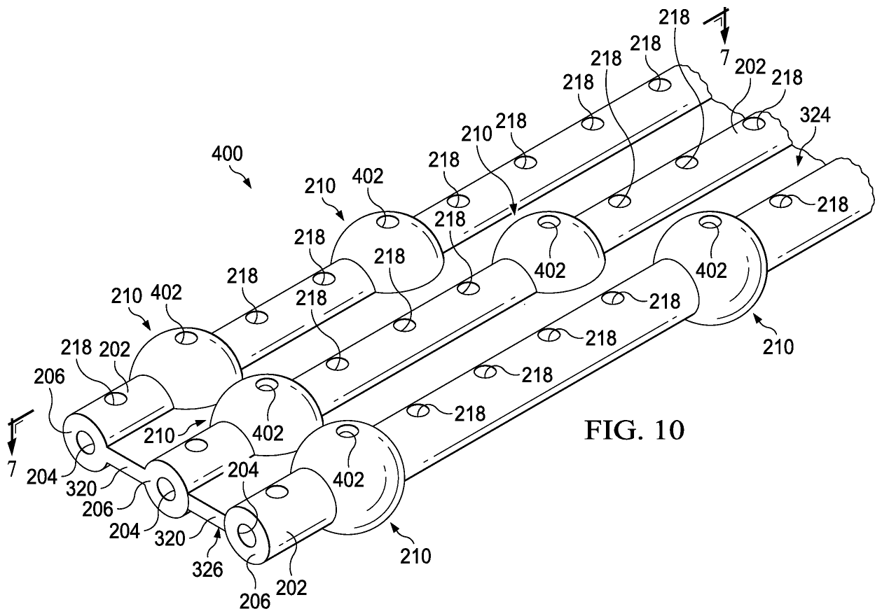

FIG. 10 is a perspective view and FIG. 11 is a plan view of another tissue interface 108, illustrating additional details that may be associated with some embodiments. The tissue interface 108 may be a wound filler 400. The wound filler 400 includes a plurality of tubes 202 joined by the membrane 320. In some embodiments, each tube 202 may have a blister 210 aligned with and adjacent to a blister 210 of an adjacent tube 202. Each tube 202 may also have a blister 210 aligned with but spaced apart from a blister 210 of an adjacent tube 202. For example, a pitch between adjacent blisters 210 can be about twice the outer diameter of an individual blister 210. Each blister 210 may have an aperture 402 disposed on a top center of the blister 210. In some embodiments, each aperture 402 can have an average effective diameter between about 0.2 mm and about 3 mm. Each aperture 402 may extend from the surface 212 through a wall of the blister 210 into the interior space 216. Each aperture 402 can provide fluid communication across the wall of the blister 210. In some embodiments, the wound filler 400 may have an aperture 402 on the first side 324 of the membrane 320. In other embodiments, the wound filler 400 may have an aperture 402 on the second side 326 of the membrane 320. In still other embodiments, the wound filler 400 may have an aperture 402 on both the first side 324 and the second side 326 of the membrane 320. The wound filler 400 can be manufactured and used as described above with respect to the wound filler 200 and the wound filler 300.

The systems, apparatuses, and methods described herein may provide significant advantages. For example, the tissue interfaces described herein are low-cost and have a high-strength. The wound fillers may not shed particulates and can treat both deep and shallow wounds. In some embodiments, the tissue interfaces can be transparent while still encouraging granulation. The tissue interfaces are also free from ingrowth issues resulting in little discomfort upon removal. The tissue interfaces can be provided in a roll to simplify deployment. In some embodiments, the tissue interfaces may be supplied as a flexible foldable sheet to reduce cutting to size operations by the user. The variation in blisters can provide offloading around sensitive wounds, and the interconnected cell designs may be used to give both a negative pressure therapy interface and a controlled positive pressure layer. For example, the blisters can bridge a tissue site to direct forces to a periwound area and not the tissue site itself.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A dressing for use with negative-pressure treatment, the dressing comprising:
a tube formed from a non-porous material, the tube having:
a first end,
a second end,
a lumen extending from the first end to the second end, and
a wall surrounding the lumen;
at least one blister in the wall of the tube, the at least one blister proximate to the second end of the tube; and
at least one aperture in the wall of the second end of the tube, the at least one aperture configured to provide fluid communication across the wall from the lumen to an exterior of the tube.

2. The dressing of claim 1, wherein the tube has an inner diameter between about 0.1 mm and about 2 mm.

3. The dressing of claim 1 or claim 2, wherein the tube has a wall thickness up to about 2 mm.

4. The dressing of any preceding claim, wherein the tube is formed from a thermoplastic.

5. The dressing of claim 4, wherein the thermoplastic comprises a polythene, a polyester, or a polyurethane.

6. The dressing of claim 1 or claim 2, wherein the tube comprises a thermoplastic elastomer.

7. The dressing of claim 6, wherein the thermoplastic elastomer comprises a styrene ethylene butadiene styrene (SEBS).

8. The dressing of claim 1 or claim 2, wherein the tube comprises an elastomeric.

9. The dressing of claim 8, wherein the elastomeric comprises a silicone.

10. The dressing of any preceding claim, wherein the tube is clear.

11. The dressing of any preceding claim, wherein the tube is colored.

12. The dressing of any preceding claim, wherein the at least one blister has an average effective diameter between about 1 mm and about 5 mm.

13. The dressing of any preceding claim, wherein the at least one blister comprises a plurality of blisters.

14. The dressing of claim 13, wherein a spacing between adjacent blisters of the plurality of blisters is equal.

15. The dressing of claim 13, wherein each blister of the plurality of blisters has a same average effective diameter.

16. The dressing of claim 13, wherein:
a first portion of the plurality of blisters has a first average effective diameter; and
a second portion of the plurality of blisters has a second average effective diameter; and
the first average effective diameter is different than the second average effective diameter.

17. The dressing of claim 16, further comprising a third portion of the plurality of blisters having a third average effective diameter, the third average effective diameter being different from the first average effective diameter and the second average effective diameter.

18. The dressing of claim 13, wherein each blister of the plurality of blisters has a different average effective diameter.

19. The dressing of any preceding claim, wherein the at least one blister is hemispherical.

20. The dressing of any of claims 1-18, wherein the at least one blister is spherical.

21. The dressing of any of claims 1-18, wherein the at least one blisters is polygonal.

22. The dressing of any of claims 1-18, wherein the at least one blister is conical.

23. The dressing of claim 1, wherein the tube comprises a plurality of tubes, the dressing further comprising:
a webbing coupling each tube to an adjacent tube; and
a plurality of perforations through the webbing.

24. The dressing of claim 23, further comprising a film coupled to the blisters, the film having a plurality of fenestrations.

25. The dressing of claim 24, wherein the film is formed from a same material as the plurality of tubes.

26. The dressing of claim 23, wherein the plurality of tubes are coplanar.

27. The dressing of claim 23, wherein the webbing further comprises at least one groove.

28. The dressing of claim 23, wherein the plurality of perforations further comprise tear lines.

29. The dressing of claim 1, wherein the tube comprises a micro-balloon.

30. The dressing of claim 1, further comprising at least one aperture disposed in the at least one blister.

31. A method of manufacturing a dressing, the method comprising:
forming a conduit from a non-porous material, the conduit having:
a proximal end,
a distal end,
a lumen extending from the proximal end to the distal end, and
a wall surrounding the lumen;
forming at least one bubble in the wall of the conduit, the at least one bubble proximate to the distal end of the conduit; and
forming at least one opening in the wall of the distal end of the conduit, the at least one opening configured to provide fluid communication across the wall from the lumen to an exterior of the conduit.

32. The method of claim 31, wherein:
forming a conduit comprises:
extruding the conduit, and
cooling the conduit; and
forming at least one bubble comprises:
placing the conduit in a mold,
heating the conduit,
applying a gas to the lumen of the conduit, and
inflating the conduit to fill the mold.

33. The method of claim 31, wherein forming a conduit comprises:
forming a plurality of conduits;
forming a plurality of mats;
coupling at least two of the plurality of conduits to each other with a mat of the plurality of mats; and
forming a plurality of perforations in the plurality of mats.

34. The method of claim 33, wherein the plurality of perforations are aligned to form tear lines between adjacent conduits of the plurality of conduits.

35. The method of claim 33, wherein the method further comprises coupling a film to the bubbles.

36. The method of claim 35, wherein coupling a film to the bubbles comprises laminating the film to the bubbles, the film having a plurality of fenestrations.

37. The method of claim 35, wherein coupling a film to the bubbles comprises:
extruding the plurality of conduits; and
co-extruding the film.

38. A dressing for use with negative-pressure treatment, the dressing having a tube formed from a non-porous material, the tube having a first end, a second end, a lumen extending from the first end to the second end, and a wall surrounding the lumen; at least one blister in the wall of the tube, the at least one blister proximate to the second end of the tube; and at least one aperture in the wall of the second end of the tube, the at least one aperture configured to provide fluid communication across the wall from the lumen to an exterior of the tube, the dressing formed by a process comprising:
extruding the tube; and
cooling the tube;
placing the tube in a mold;
heating the tube;
applying a gas to the lumen of the tube; and
inflating the tube to fill the mold.

39. The dressing of claim 38, wherein extruding a tube comprises:
extruding a plurality of tubes;
extruding a plurality of mats;
coupling at least two of the plurality of tubes to each other with a mat of the plurality of mats; and
forming a plurality of perforations in the plurality of mats.

40. The dressing of claim 39, wherein the plurality of perforations are aligned to form tear lines between adjacent tubes of the plurality of tubes.

41. The dressing of claim 39, wherein the process further comprises coupling a membrane to the blisters.

42. The dressing of claim 41, wherein coupling a film to the blisters comprises laminating the film to the blisters, the film having a plurality of fenestrations.

43. The dressing of claim 39, wherein coupling a film to the blisters comprises:
extruding the plurality of tubes; and
co-extruding the film.

44. The systems, apparatuses, and methods substantially as described herein.
